(19) United States
(12) Patent Application Publication
(10) Pub. No.: US 2008/0177392 A1
(43) Pub. Date: Jul. 24, 2008

(54) Closed system artificial intervertebral disc

(76) Inventors: Michael S. Williams, Santa Rosa, CA (US); Jeffrey A. Smith, Petaluma, CA (US); Daniel W. Fifer, Windsor, CA (US); Richard A. Glenn, Santa Rosa, CA (US); Geoffrey A. Orth, Sebastopol, CA (US)

Correspondence Address: DEANNA J. SHIRLEY, 3418 BALDWIN WAY, SANTA ROSA, CA, 95403, US

(21) Appl. No.: 12/004,322

(22) Filed: Dec. 19, 2007

Related U.S. Application Data
(63) Continuation-in-part of application No. 11/216,581, filed on Aug. 30, 2005, now abandoned.

(51) Int. Cl. A61F 2/44

(52) U.S. Cl. 623/17.16; 623/17.12

(57) ABSTRACT

An artificial intervertebral disc and disc nucleus are described herein having chambers and dampening members. The dampening members may be within or outside of the main body of the device. The chambers may be filled with a suitable liquid, gas, or both, and separated by valves to regulate flow of fluid between chambers, within a dampening member, between the main body and dampening member, or all of the above. Chambers may be filled with responsive hydrogels, EPAM, or other suitable materials, and the device may have activation plates or members, a strain gauge, a pressure sensor, or other means for detecting changes in the materials and/or triggering desired changes in the materials in order to mimic the behavior of a healthy native disc or disc nucleus. A control system may be in communication with the device for receiving feedback and delivering stimuli to initiate desired changes in the fluids or other materials. Membranes may be of variable permeability and may be metallized to ensure as low permeability as possible. Dampening members may be filled during manufacture with carbon dioxide or other suitable gas which may be in a supercritical state and allowed to return to ambient temperature and gaseous state or by other means. Methods of manufacture, delivery of the artificial disc and related structures, and methods of treatment are also described.

### RELATED APPLICATIONS

This application is related to U.S. application Ser. No. 11/216,581, filed Aug. 30, 2005, titled “Closed System Artificial Disc”, by Smith, et al.; U.S. Application Ser. No. 60/611,161 titled “Closed System Artificial Intervertebral Disc”, by Smith, et al., filed Sep. 17, 2004, and the entirety of both is hereby incorporated as if fully set forth herein.

### FIELD OF THE INVENTION

The invention herein relates generally to medical devices and methods of treatment, and more particularly to devices and methods used in the treatment of a degenerated or traumatized intervertebral disc.

### BACKGROUND OF THE INVENTION

Intervertebral disc degeneration is a leading cause of pain and disability, occurring in a substantial majority of people at some point during adulthood. The intervertebral disc, comprising primarily the nucleus pulposus and surrounding annulus fibrosus, constitutes a vital component of the functional spinal unit. The intervertebral disc maintains space between adjacent vertebral bodies, absorbs impact between and cushions the vertebral bodies. The disc allows for fluid movement between the vertebral bodies, both subtle (for example, with each breath inhaled and exhaled) and dramatic (including rotational movement and bending movement in all planes.) Deterioration of the biological and mechanical integrity of an intervertebral disc as a result of disease and/or aging may limit mobility and produce pain, either directly or indirectly as a result of disruption of spinal function. Estimated health care costs of treating disc degeneration in the United States exceed $60 billion annually.

Age-related disc changes are progressive, and, once significant, increase the risk of related disorders of the spine. The degenerative process alters intradiscal pressures, causing a relative shift of axial load-bearing to the peripheral regions of the endplates and facets of the vertebral bodies. Such a shift promotes abnormal loading of adjacent intervertebral discs and vertebral bodies, altering spinal balance, shifting the axis of rotation of the vertebral bodies, and increasing risk of injury to these units of the spine. Further, the transfer of biomechanical loads appears to be associated with the development of other disorders, including both facet and ligament hypertrophy, osteophyte formation, lyphosis, spondylolisthesis, nerve damage, and pain.

In addition to age-related changes, numerous individuals suffer trauma-induced damage to the spine including the intervertebral discs. Trauma induced damage may include ruptures, tears, prolapse, herniations, and other injuries that cause pain and reduce strength and function.

Non-operative therapeutic options for individuals with neck and back pain include rest, analgesics, physical therapy, heat, and manipulation. These treatments fail in a significant number of patients. Current surgical options for spinal disease include discectomy, discectomy combined with fusion, and fusion alone. Numerous discectomies are performed annually in the United States. The procedure is effective in promptly relieving significant radicular pain, but, in general, the return of pain increases proportionally with the length of time following surgery. In fact, the majority of patients experience significant back pain by ten years following lumbar discectomy.

An attempt to overcome some of the possible reasons for failure of discectomy, fusion has the potential to maintain normal disc space height, to eliminate spine segment instability, and eliminate pain by preventing motion across a destabilized or degenerated spinal segment.

However, although some positive results are possible, spinal fusion may have harmful consequences as well. Fusion involves joining portions of adjacent vertebrae to one another. Because motion is eliminated at the treated level, the biomechanics of adjacent levels are disrupted. Resulting pathological processes such as spinal stenosis, disc degeneration, osteophyte formation, and others may occur at levels adjacent to a fusion, and cause pain in many patients. In addition, depending upon the device or devices and techniques used, surgery may be invasive and require a lengthy recovery period.

Consequently, there is a need in the art to treat degenerative disc disease and/or traumatized intervertebral discs, while eliminating the shortcomings of the prior art. There remains a need in the art to achieve the benefit of removal of a non-functioning intervertebral disc, to replace all or a portion of the disc with a device that will function as a healthy disc, eliminating pain, while preserving motion and maintaining disc height. There remains a need for an artificial disc or other device that maintains the proper intervertebral spacing, allows for motion, distributes axial load appropriately, and provides stability. comprise the characteristic lower durometer than the annulus fibrosus, must mimic the behavior of a healthy native nucleus upon load increase and decrease, and the annulus fibrosus must comprise the requisite stiffness as compared with the nucleus. Further, there remains a need for an artificial disc that can withstand typical cyclic stresses and perform throughout the life a patient. An artificial disc that can be implanted using minimally invasive techniques is also needed And finally, a device that is compatible with current imaging modalities, such as Magnetic Resonance Imaging (MRI) is needed.

### SUMMARY OF THE INVENTION

An artificial disc or disc nucleus having a first membrane and a second membrane defining a first chamber and dampening members and filled with fluid is disclosed. The first and second membranes are substantially impermeable, and may have a metallized coating. A third membrane that is permeable and defines a third chamber substantially surrounding the dampening members is also disclosed. One or more compressible gases may fill a chamber or a dampening member. The device may be filled with a responsive hydrogel or EPAM.

An artificial disc or disc nucleus according to the invention may have one or more activation members in communication with a fluid within the device. The artificial disc or disc nucleus may have one or more sensors for detecting a change in one or more physical or chemical characteristics of one or more of said fluids and a control system. One or more physical or chemical characteristics may be volume, compression, density, strain, temperature, pH, salts concentration, electrical potential, and hydration. The control system may deliver electrical charge, radiofrequency, ultrasound, and heat.

The dampening members may have one or more valves for regulating the flow of one or more fluids within the dampening member. The artificial disc or disc nucleus may have one or more valves disposed between the body and the one or more dampening members for regulating the flow of fluid. The damping members may have one or more chambers and the membranes may have compliant regions and rigid regions.

A method of manufacture of an artificial disc or disc nucleus may include the steps of preparing a first polymeric membrane; forming a body with an interior and one or more dampening members from said membrane. The method may include with the added step of introducing said one or more dampening members into the interior of the body.

The dampening members may be prepared by forming an enclosed member from said first membrane; introducing a compressible gas in a supercritical state into said member; and allowing said compressible gas to return to ambient temperature to form a dampening member. The method may include the added steps of preparing a second polymeric membrane that is permeable and substantially enclosing one or more dampening members with the second membrane. The method may also include the steps of introducing fluid in the body, and the fluid may be a responsive hydrogel or EPAM.

The method may also include providing a valve within a dampening member, an additional membrane, a partition, and/or metallizing the dampening member either prior to or subsequent to the introduction of a compressible gas. It may also include adding sensors and/or a control system to the device.

### BRIEF DESCRIPTION OF THE DRAWINGS

FIG. 1 is a perspective view of an embodiment according to the invention in “see-through” mode.

FIG. 2 is a “cut-away” view of the embodiment of FIG. 1.

FIG. 3 is a side view of one of the internal components of the embodiment of FIGS. 1 and 2.

FIG. 4A is a “cut-away” view of an alternative embodiment of an internal component illustrated in an “at rest” configuration.

FIG. 4B is a “cut-away” view of the embodiment illustrated in FIG. 4A in an “under load” configuration.

FIG. 5 is a perspective view of an alternative embodiment according to the invention in “see-through” mode.

FIG. 6 is a “cut-away” view of the embodiment of FIG. 5.

FIG. 7 is a perspective view of one of the internal components of the embodiment of FIGS. 5 and 6.

FIG. 8 is a “cut-away” view of the component illustrated in FIG. 7

FIG. 9 is a perspective view of an internal component of another alternative embodiment according to the invention illustrated in an “at rest” configuration.

FIG. 10 is the internal component illustrated in FIG. 9, following application of a load.

FIG. 11 is a side view of an internal component of yet another alternative embodiment according to the invention illustrated in an “at rest” configuration.

FIG. 12 is the internal component illustrated in FIG. 11, following application of a load.

FIG. 13 is a cross-sectional side view of yet another alternative embodiment according to the invention.

FIG. 14 is a cross-sectional side view of still another alternative embodiment according to the invention.

FIG. 15 is a perspective view of yet another alternative embodiment according to the invention shown in “see-through” mode within a delivery device.

FIG. 16 illustrates in a plan view the position of an embodiment according to the invention in relation to a vertebra of a subject while undergoing percutaneous delivery to a subject, the vertebra shown in a cross-sectional plan view.

FIG. 17 is a plan view of an embodiment according to the invention, at a later step in the delivery sequence, with the delivery device illustrated in “see-through” mode.

FIG. 18 is a plan view of an embodiment according to the invention at a subsequent step in the delivery sequence.

FIG. 19 illustrates a perspective view of an embodiment according to the invention following deployment.

FIG. 20 illustrates area “A” of FIG. 19 in a greater level of detail.

### DETAILED DESCRIPTION OF THE INVENTION

An endoprosthesis known as an artificial disc nucleus, or an artificial disc are designed to replace a degenerated intervertebral disc nucleus, disc annulus, or both. Such an artificial disc annulus, disc nucleus or disc may be expandable and/or self-expanding.

An “expandable” endoprosthesis comprises a reduced profile configuration and an expanded profile configuration. An expandable endoprosthesis according to the invention may undergo a transition from a reduced configuration to an expanded profile configuration via any suitable means, or may be self-expanding. Some embodiments according to the invention may comprise a substantially hollow interior that may be filled with a suitable medium, examples of which are set forth below. Such embodiments may accordingly be introduced into the body in a collapsed configuration, and, following introduction, may be filled to form a deployed configuration. Embodiments according to the invention may accordingly be implanted percutaneously or surgically. If implanted surgically, embodiments according to the invention may be implanted from either an anterior or a posterior approach, following the removal of some or all of the native disc, excepting the periphery of the native nucleus.

“Preservation of mobility” refers to the desired maintenance of normal motion between separate spinal segments.

“Spinal unit” refers to a set of the vital functional parts of the spine including a vertebral body, endplates, facets, and intervertebral disc.

The term “cable” refers to any generally elongate member fabricated from any suitable material, whether polymeric, metal or metal alloy, natural or synthetic.

The term “fiber” refers to any generally elongate member fabricated from any suitable material, whether polymeric, natural or synthetic, metal or metal alloy.

As used herein, the term “braid” refers to any braid or mesh or similar wound or woven structure produced from between 1 and several hundred longitudinal and/or transverse elongate elements wound, woven, braided, knitted, helically wound, or intertwined by any manner, at angles between 0 and 180 degrees and usually between 45 and 105 degrees, depending upon the overall geometry and dimensions desired.

Unless specified, suitable means of attachment may include by thermal melt, chemical bond, adhesive, sintering, welding, or any means known in the art.

As used herein, a device is “implanted” if it is placed within the body either temporarily or to remain for any length of time following the conclusion of the procedure to place the device within the body.

The term “diffusion coefficient” refers to the rate by which a substance elutes, or is released either passively or actively from a substrate.

Unless specified, suitable means of attachment may include by thermal melt, chemical bond, adhesive, sintering, welding, or any means known in the art.

“Shape memory” refers to the ability of a material to undergo structural phase transformation such that the material may define a first configuration under particular physical and/or chemical conditions, and to revert to an alternate configuration upon a change in those conditions. Shape memory materials may be metal alloys including but not limited to nickel titanium, or may be polymeric. A polymer is a shape memory polymer if the original shape of the polymer is recovered by heating it above a shape recovering temperature (defined as the transition temperature of a soft segment) even if the original molded shape of the polymer is destroyed mechanically at a lower temperature than the shape recovering temperature, or if the memorized shape is recoverable by application of another stimulus. Such other stimulus may include but is not limited to pH, salinity, hydration, radiation, including but not limited to radiation in the ultraviolet range, and others. Some embodiments according to the invention may comprise one or more polymers having a structure that assumes a first configuration, a second configuration, and a hydrophilic polymer of sufficient rigidity coated upon at least a portion of the structure when the device is in the second configuration. Upon placement of the device in an aqueous environment and consequent hydration of the hydrophilic polymer, the polymer structure reverts to the first configuration.

Some embodiments according to the invention, while not technically having shape memory characteristics, may nonetheless readily convert from a constrained configuration to a deployed configuration upon removal of constraints, as a result of a material's elasticity, super-elasticity, a particular method of “rolling down” and constraining the device for delivery, or a combination of the foregoing. Such embodiments may comprise one or more elastomeric or rubber materials.

As used herein, the term “segment” refers to a block or sequence of polymer forming part of the shape memory polymer. The terms hard segment and soft segment are relative terms, relating to the transition temperature of the segments. Generally speaking, hard segments have a higher glass transition temperature than soft segments, but there are exceptions.

“Transition temperature” refers to the temperature above which a shape memory polymer reverts to its original memorized configuration.

The term “strain fixity rate” R_{f} is a quantification of the fixability of a shape memory polymer's temporary form, and is determined using both strain and thermal programs. The strain fixity rate is determined by gathering data from heating a sample above its melting point, expanding the sample to 200% of its temporary size, cooling it in the expanded state, and drawing back the extension to 0%, and employing the mathematical formula:

R_{f}(N)=εᵤ(N)/εₘ

where εᵤ(N) is the extension in the tension-free state while drawing back the extension, and εₘ is 200%.
The “strain recovery rate” Rᵣ describes the extent to which the permanent shape is recovered:

$${R}_{r} ( N ) = \frac{{ɛ}_{m} - {ɛ}_{p} ( N )}{{ɛ}_{m} - {ɛ}_{p} ( N - 1 )}$$

where εₚ is the extension at the tension free state.

A “switching segment” comprises a transition temperature and is responsible for the shape memory polymer's ability to fix a temporary shape.

A “thermoplastic elastomer” is a shape memory polymer having crosslinks that are predominantly physical crosslinks.

A “thermoset” is a shape memory polymer having a large number of crosslinks that are covalent bonds.

Shape memory polymers are highly versatile, and many of the advantageous properties listed above are readily controlled and modified through a variety of techniques. Several macroscopic properties such as transition temperature and mechanical properties can be varied in a wide range by only small changes in their chemical structure and composition. More specific examples are set forth in U.S. patent application Ser. No. 10/988,814 and are incorporated in their entirety as if fully set forth herein.

Shape memory polymers are characterized by two features, triggering segments having a thermal transition Tₜᵣₐₙₛ within the temperature range of interest, and crosslinks determining the permanent shape. Depending on the kind of crosslinks (physical versus covalent bonds), shape memory polymers can be thermoplastic elastomers or thermosets. By manipulating the types of crosslinks, the transition temperature, and other characteristics, shape memory polymers can be tailored for specific clinical applications.

More specifically, according the invention herein, one can the control shape memory behavior and mechanical properties of a shape memory polymer through selection of segments chosen for their transition temperature, and mechanical properties can be influenced by the content of respective segments. The extent of crosslinking can be controlled depending on the type of material desired through selection of materials where greater crosslinking makes for a tougher material than a polymer network. In addition, the molecular weight of a macromonomeric crosslinker is one parameter on the molecular level to adjust crystallinity and mechanical properties of the polymer networks. An additional monomer may be introduced to represent a second parameter.

Further, the annealing process (having heating of the materials according to chosen parameters including but not limited to time and temperature) increases polymer chain crystallization, thereby increasing the strength of the material. Consequently, according to the invention, the desired material properties can be achieved by using the appropriate ratio of materials and by annealing the materials.

In addition, polymers are a suitable material when different degrees of permeability are desired in different components of a device or in alternative embodiments according to an invention. The relative permeability of polymeric membranes may be adjusted according to the demands of a particular component of the invention. Some embodiments according to the invention herein comprise relatively permeable outer membranes. Some permeability in an outer membrane may be desired, for example, to allow for the diffusion of water into and out of the device. In addition, internal components which serve to absorb the impact of a load may have an outer membrane which is somewhat permeable to allow for the diffusion of a hydrogel into and out of the component. Such membranes may be constructed from, for example, Chronoflex AR®, or an aromatic polyurethane. Extent of crystallization, density, and other properties may be tailored during the preparation of the membrane according to the desired permeability. Permeability may be enhanced by lasing porosity through a membrane, by an expanding and processing method as used to prepare, for example, expanded polytetrafluoroethylene, by mixing one or more salts in the polymer and allowing to dissolve out of the membrane, or through a process known as phase inversion, in which uncured polymer is placed in water thereby creating a porous scaffold for later processing steps, or other suitable methods known in the art. A membrane used in the construction of a component of a device according to the invention which is described as relatively, somewhat, or substantially permeable may be prepared as set forth above or according to an suitable method or material.

Alternative embodiments, and/or separate components of a device according to the invention may be constructed from substantially impermeable polymers. Accordingly, such embodiments may comprise a fluid retention body having a membrane layer having relatively low level permeability, having, for example, polyvinyl chloride (PVC), polyurethane, and/or laminates of polyethylene terephthalate (PET) or nylon fibers or films within layers of PVC, or other suitable material. Such a fluid retention body or membrane layer alternatively may comprise Kevlar, polyimide, a suitable metal, or other suitable material within layers of PVC, polyurethane or other suitable material. Such laminates may be of solid core, braided, woven, wound, or other fiber mesh structure, and provide stability, strength, and a controlled degree of compliance. Such a laminate membrane layer may be manufactured using radiofrequency or ultrasonic welding, adhesives including ultraviolet curable adhesives, or thermal energy.

An impermeable membrane constructed from the materials set forth as examples above may be used to construct a spherical component that is filled with carbon dioxide or other suitable gas. Permeability may be further decreased, to prevent diffusion of the gas, for example, in a polyurethane that has been metallized, or coated with a pure metal, such as, for example, titanium, aluminum or platinum. Such a metal may be applied via a vapor deposition process performed in a vacuum following construction of the sphere and filling with carbon dioxide, which may be in a supercritical state, and then allowed to return to ambient temperature. Numerous technologies known in the art and available commercially, such as, for example from VacuCoat Technologies, Inc., of Clinton Township, Michigan are acceptable. Membranes used in the construction of a component of an embodiment according to the invention which are described as relatively low permeability or as impermeable may be prepared as set forth above or according to other suitable means and materials.

Additionally, the properties of polymers can be enhanced and differentiated by controlling the degree to which the material crystallizes through strain-induced crystallization. Means for imparting strain-induced crystallization are enhanced during deployment of an endoprosthesis according to the invention. Upon expansion of an endoprosthesis according to the invention, focal regions of plastic deformation undergo strain-induced crystallization, further enhancing the desired mechanical properties of the device, such as further increasing strength. The strength is optimized when the endoprosthesis is induced to bend preferentially at desired points.

Natural polymer segments or polymers include but are not limited to proteins such as casein, gelatin, gluten, zein, modified zein, serum albumin, and collagen, and polysaccharides such as alginate, chitin, celluloses, dextrans, pullulane, and polyhyaluronic acid; poly(3-hydroxyalkanoate)s, especially poly(.beta-hydroxybutyrate), poly(3-hydroxyoctanoate) and poly(3-hydroxyfatty acids).

Suitable synthetic polymer blocks include polyphosphazenes, poly(vinyl alcohols), polyamides, polyester amides, poly(amino acid)s, synthetic poly(amino acids), polycarbonates, polyacrylates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyesters, polyethylene terephthalate, polysiloxanes, polyurethanes, fluoropolymers (including but not limited to polyfluorotetraethylene), and copolymers thereof.

Examples of suitable polyacrylates include poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate) and poly(octadecyl acrylate).

Synthetically modified natural polymers include cellulose derivatives such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, arboxymethyl cellulose, cellulose triacetate and cellulose sulfate sodium salt. These are collectively referred to herein as “celluloses”.

For those embodiments having a shape memory polymer, the degree of crystallinity of the polymer or polymeric block(s) is between 3 and 80%, more often between 3 and 65%. The tensile modulus of the polymers below the transition temperature is typically between 50 MPa and 2 GPa (gigapascals), whereas the tensile modulus of the polymers above the transition temperature is typically between 1 and 500 MPa. Most often, the ratio of elastic modulus above and below the transition temperature is 20 or more.

The melting point and glass transition temperature of the hard segment are generally at least 10 degrees C., and preferably 20 degrees C., higher than the transition temperature of the soft segment. The transition temperature of the hard segment is preferably between −60 and 270 degrees C., and more often between 30 and 150 degrees C. The ratio by weight of the hard segments to soft segments is between about 5:95 and 95:5, and most often between 20:80 and 80:20. The shape memory polymers contain at least one physical crosslink (physical interaction of the hard segments) or contain covalent crosslinks instead of a hard segment. The shape memory polymers can also be interpenetrating networks or semi-interpenetrating networks. A typical shape memory polymer is a block copolymer.

Examples of suitable hydrophilic polymers include but are not limited to poly(ethylene oxide), polyvinyl pyrrolidone, polyvinyl alcohol, poly(ethylene glycol), polyacrylamide poly(hydroxy alkyl methacrylates), poly(hydroxy ethyl methacrylate), hydrophilic polyurethanes, HYPAN, oriented HYPAN, poly(hydroxy ethyl acrylate), hydroxy ethyl cellulose, hydroxy propyl cellulose, methoxylated pectin gels, agar, starches, modified starches, alginates, hydroxy ethyl carbohydrates and mixtures and copolymers thereof.

Hydrogels can be formed from polyethylene glycol, polyethylene oxide, polyvinyl alcohol, polyvinyl pyrrolidone, polyacrylates, poly (ethylene terephthalate), poly(vinyl acetate), poly-hema hydroxyethyl methacrylate, and copolymers and blends thereof. Several polymeric segments, for example, acrylic acid, are elastomeric only when the polymer is hydrated and hydrogels are formed. Other polymeric segments, for example, methacrylic acid, are crystalline and capable of melting even when the polymers are not hydrated. Either type of polymeric block can be used, depending on the desired application and conditions of use.

Responsive or “smart” hydrogels are capable of dramatic dimensional alterations from swelling or shrinkage in response to an environmental trigger, such as, for example, change in temperature, pH, ionic strength, salt type(s), electric charge, solvent type, etc. Such a hydrogel may be incorporated into a device according to the invention in order to confer the ability to mimic a natural disc and/or disc nucleus. For example, a disc or disc nucleus may undergo compression throughout the day, and rehydrate and/or expand at rest. Device performance may be actively or passively induced according to the particular environmental factor selected. A reconfiguration from temperature change may be induced by, for example, heat pack or ice pack.

A control system may be coupled with a device, either integrally with or separate from a device. Such a control system may be hard-wired to the device or selected components of the device, or may communicate with the device through wireless means, such as, for example, by radio frequency or induction. Electric charge or other environmental stimuli may be delivered to the device via the control system. In addition, a device may include one or more sensors, such as, for example, a strain gauge, which provides feedback to the control system. Alternatively, or in addition, the control system may follow a selected time-based cycle.

An “Electroactive Polymer Artificial Muscle” (hereinafter referred to as EPAM) may be used as a material in a device according to the invention. EPAM is an electrically excitable polymer which can be activated to shrink or swell in response to an electrical stimulus. In addition, EPAM creates a voltage potential when either compressed or elongated, thereby generating electrical potential that can in turn stimulate a second component (either an additional EPAM component or electrically responsive hydrogel) or can be stored within the device's control system (such as, for example, in a rechargeable battery or a capacitor.)

Examples of highly elastic materials including but not limited to vulcanized rubber, polyurethanes, thermoplastic elastomers, and others may be used according to the invention.

Curable materials include any material capable of being able to transform from a fluent or soft material to a harder material, by cross-linking, polymerization, or other suitable process. Materials may be cured over time, thermally, chemically, or by exposure to radiation. For those materials that are cured by exposure to radiation, many types of radiation may be used, depending upon the material. Wavelengths in the spectral range of about 100-1300 nm may be used. The material should absorb light within a wavelength range that is not readily absorbed by tissue, blood elements, physiological fluids, or water. Ultraviolet radiation having a wavelength ranging from about 100-400 nm may be used, as well as visible, infrared and thermal radiation. The following materials are some examples of curable materials: urethanes, polyurethane oligomer mixtures, acrylate monomers, aliphatic urethane acrylate oligomers, acrylamides, UV curable epoxies, photopolymerizable polyanhydrides and other UV curable monomers. Alternatively, the curable material can be a material capable of being chemically cured, such as silicone based compounds which undergo room temperature vulcanization.

Though not limited thereto, some embodiments according to the invention comprise one or more therapeutic substances that will elute from the surface. Suitable therapeutics include but are not limited to bone growth accelerators, bone growth inducing factors, osteoinductive agents, immunosuppressive agents, steroids, anti-inflammatory agents, pain management agents (e.g., analgesics), tissue proliferative agents to enhance regrowth and/or strengthening of native disc materials, and others. According to the invention, such surface treatment and/or incorporation of therapeutic substances may be performed utilizing one or more of numerous processes that utilize carbon dioxide fluid, e.g., carbon dioxide in a liquid or supercritical state. A supercritical fluid is a substance above its critical temperature and critical pressure (or “critical point”).

The use of polymeric materials in the fabrication of endoprostheses confers the advantages of improved flexibility, compliance and conformability. Fabrication of an endoprosthesis according to the invention allows for the use of different materials in different regions of the prosthesis to achieve different physical properties as desired for a selected region. An endoprosthesis having polymeric materials has the additional advantage of compatibility with magnetic resonance imaging, potentially a long-term clinical benefit.

As set forth above, some embodiments according to the invention may comprise components that have a substantially hollow interior that may be filled after being delivered to a treatment site with a suitable material in order to place the device in a deployed configuration A fluid retention body as set forth above may be filled with any suitable material including but not limited to saline, contrast media, hydrogels, polymeric foam, compressible gas, or any combination thereof. A polymeric foam may comprise a polyurethane intermediate having polymeric diisocyanate, polyols, and a hydrocarbon, or a carbon dioxide gas mixture. Such a foam may be loaded with any of numerous solid or liquid materials known in the art that confer radiopacity.

Such a fluid retention membrane and/or body may be designed to replace an entire intervertebral disc. Alternatively, it may replace only the nucleus pulposus or only the annulus fibrosus. Such a device may comprise one or more filling ports, and include separate filling ports for portions of the nucleus pulposus, to allow for varying durometers, and possibly varied materials in order to mimic the properties of the native disc components.

Such a device may comprise a single unit, or may be two or more individual parts. If the device comprises two or more component parts, the parts may fit together in a puzzle-like fashion. The device may further comprise alignment tabs for stable alignment between the vertebral bodies.

Such a fluid retention membrane and/or body may comprise interbody connections and/or baffles and/or partitions or generally vertically oriented membranes in order to maintain structural integrity after filling, to increase the devices ability to withstand compressive, shear, and other loading forces, and/or to direct filling material flow and positioning, and/or to partition portions of the disc in order to separate injection of different types or amounts of filling materials.

Following surgical or minimally invasive surgical access and removal of all or a portion of the native disc, a deflated fluid retention body or membrane may be delivered to the intervertebral space surgically or through a catheter and/or cannula. For example, a nuclectomy may be performed to remove the native disc nucleus and leave the native annulus intact. The access site through the native annulus may then be used to position a cannula or other suitable delivery device. Once the device is pushed out of the cannula, the membrane and/or body is positioned within the intervertebral space. The cannula can then be removed and replaced with a filling syringe or other device suitable for introducing a fill material. The membrane inflation port or ports are then attached to the injection source. Filling material is then injected and the device may unroll to fill the disc or disc nucleus space. Following injection of the filling material, which may be curable by any suitable means or may be catalytically activated or may remain in fluid form, the injection source is detached and removed.

Details of the invention can be better understood from the following descriptions of specific embodiments which are set forth as examples of the general principles of the invention. It will be appreciated that numerous structural and material modifications may be made without departing from the spirit and scope of the invention. It will also be appreciated that the following embodiments may serve as an artificial disc nucleus, artificial disc annulus, or both.

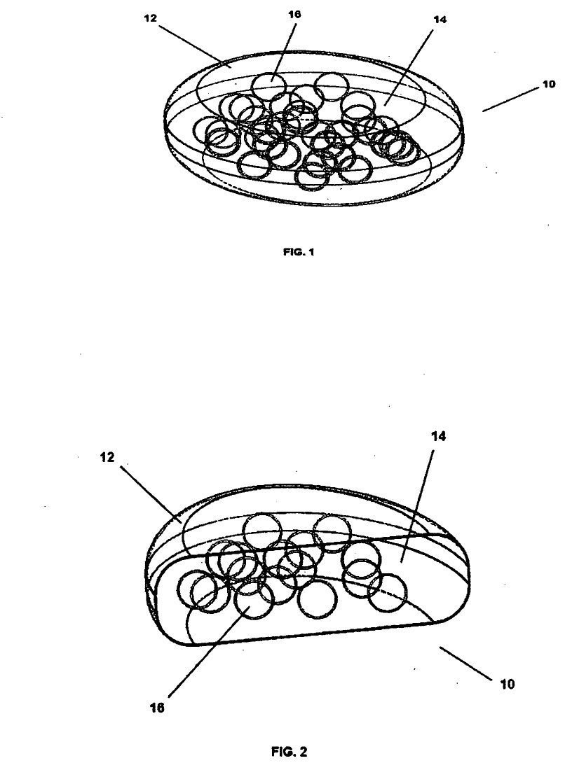

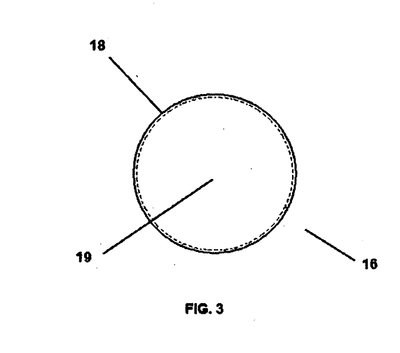

FIG. 1 illustrates an embodiment according to the invention in a deployed configuration. Disc nucleus 10 comprises substantially impermeable membrane 12 which is filled with polymer gel 14. In addition, one or more, but likely numerous dampening members, in this example, spheres 16 also fill the interior of nucleus 10. Spheres 16, which may be microspheres, and most typically are compliant to compress under a load and expand following removal of a load, can be better seen in FIGS. 2 and 3. The cut-away view of FIG. 2 illustrates spheres 16 which occupy the interior of nucleus 10.

Sphere 16, illustrated singly in FIG. 3, comprise membrane 18 that is substantially impermeable to polymer gel 14. (Gel 14 may be a hydrogel such as, for example, polyethylene glycol, PVP, or poly-hema hydroxyethyl methacrylate. Alternatively, gel 14 may be silicone.) As described above, membrane 18 may be metallized to comprise coating 19 to further decrease permeability of membrane 18. Sphere 16 may be filled with carbon dioxide in a supercritical state, then brought back to ambient temperature to form a compressible gas. Upon application of a load, sphere 16 may compress to a smaller volume, absorbing the impact of a load, thereby mimicking a healthy native disc nucleus. Following release of a load, sphere 16 may then return to its original volume. The foregoing cycle may be repeated innumerably throughout the life cycle of disc nucleus 10.

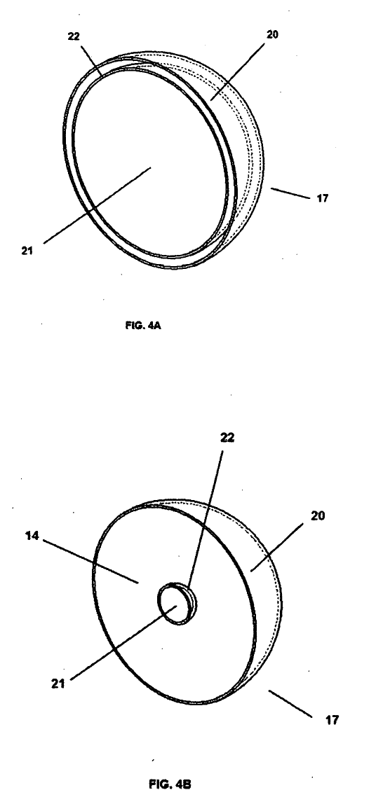

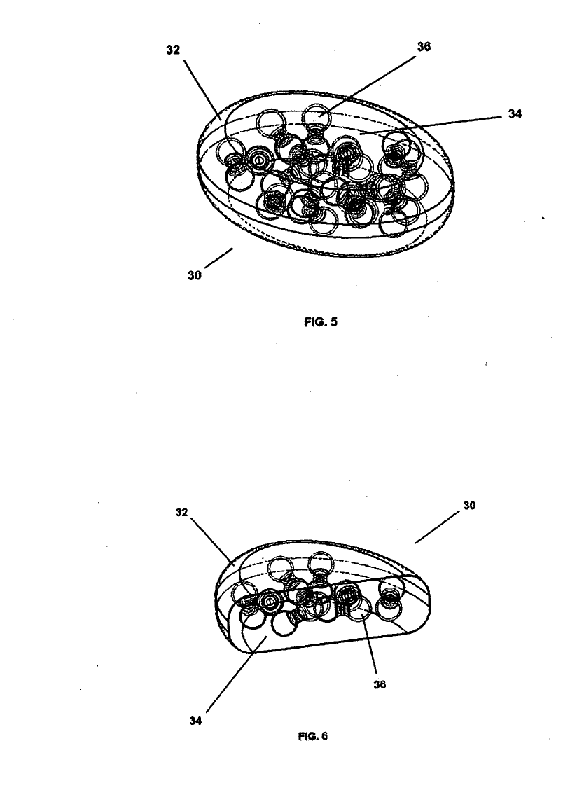

In an alternative embodiment according to the invention illustrated in FIGS. 4A and 4B, dampening member spheres 17 comprise outer membrane 20. Outer membrane 20 is relatively permeable. Spheres 17 also comprise inner membrane 20 that is substantially impermeable and defines second chamber 21. Second chamber 21 is filled with compressible carbon dioxide, or other suitable gas (not pictured). While inner membrane 22 is illustrated as resting apart from outer membrane 20 in FIG. 4A, inner membrane may in fact be fully in contact with outer membrane 20 when sphere 17 is in a steady state, or is at rest.

As illustrated in FIG. 4B, upon the application of a force, gel 14 enters permeable membrane 20. Because inner membrane 22 is substantially impermeable, compressible gas (not pictured), and consequently second chamber 21, are compressed to a smaller volume. Sphere 17 thereby mimics the behavior of a healthy native disc, and absorbs the impact of the load. Following removal of a load, the carbon dioxide or other suitable gas can expand to its pre-load volume, and gel 14 can exit outer membrane 20. Second chamber 21 will return to its pre-load or equilibrium volume. Similar to sphere 16 described above, sphere 17 can repeatedly undergo the foregoing cycle.

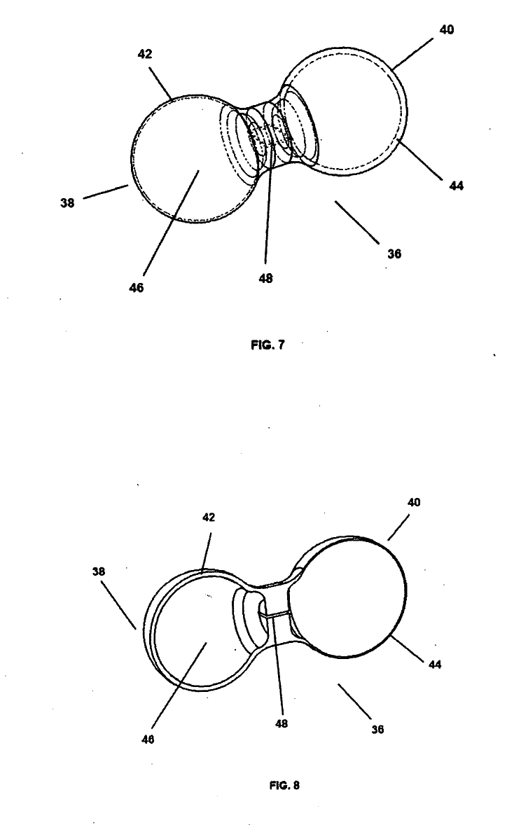

FIG. 5 illustrates an alternative embodiment according to the invention. Disc nucleus 30 comprises nuclear membrane 32 and is filled with polymer gel 34. Membrane 32 may have any level of permeability within a desired range. In addition, nucleus 30 comprises one or more, and likely a plurality of dampening members or load absorption units 36. Units 36 can be more clearly seen in FIGS. 6-8.

Unit 36 comprises first end 38 and second end 40. First end 38 comprises substantially impermeable and somewhat compliant membrane 42. Second end 40 comprises relatively rigid impermeable membrane 44. Unit 36 comprises fluid 46 within its interior and valve 48 disposed within its interior between first end 38 and second end 40. Upon application of a load, first end 38 is compressed, forcing fluid 46 through valve 48 and into second end 40. Following release of a load, compliant membrane 42 will return to its at rest configuration, and fluid 46 will flow back through valve 48 and into first end 38. Upon subsequent applications of a load, the cycle will repeat, thereby absorbing load applied, and collectively, a plurality of units 36 within membrane 32 will define disc 30 perform the function of a healthy native disc nucleus. In the alternative, a larger scale version of such a unit alone may function as an artificial disc nucleus.

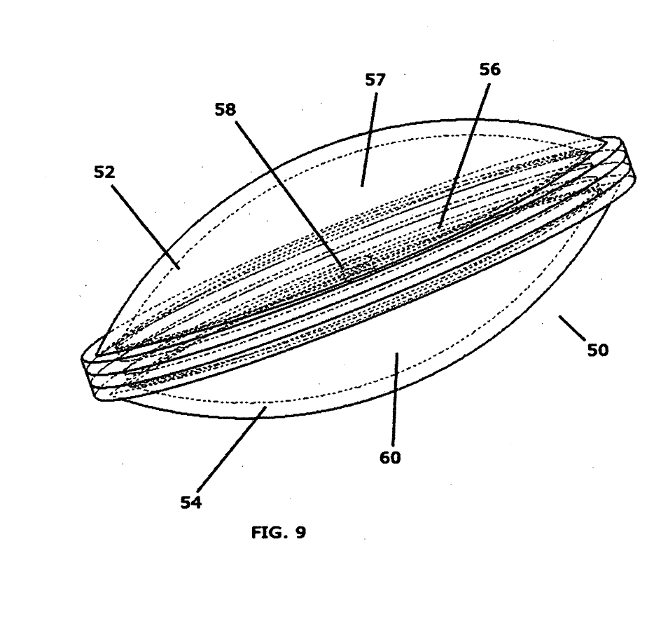

An alternative embodiment of a dampening member or load absorption unit is illustrated in FIG. 9. Load absorption unit 50 comprises relatively compliant membrane 52, relatively rigid membrane 54, valve plate 56 disposed in its substantially hollow interior 57, and valve 58 disposed within valve plate 56. Unit 50 also comprises fluid 60 within interior 57. An artificial disc or disc nucleus according to the invention similar to that described in relation to FIGS. 1 and 5 may comprise one or more, and most often a plurality of units 50 within its interior, alone or in conjunction with a fluid or gel (not pictured).

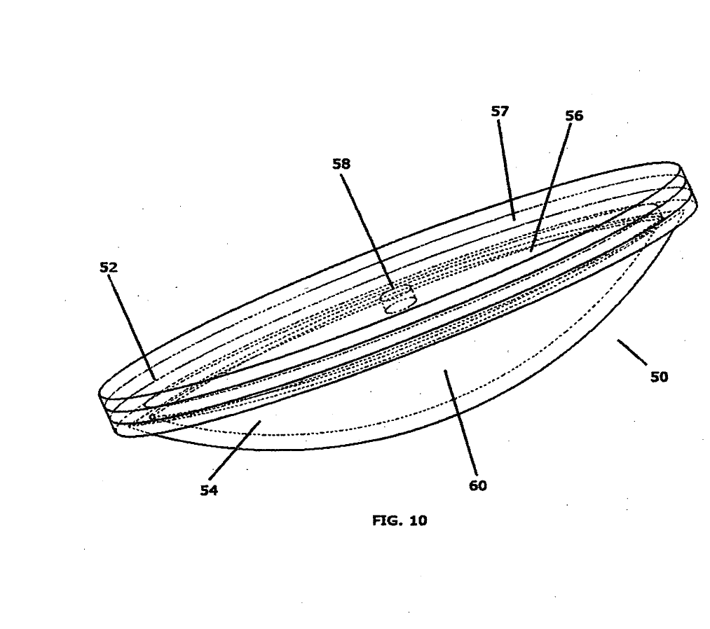

Upon application of a load, membrane 52 of unit 50 will compress, and fluid 60 will be driven through valve 58, as illustrated in FIG. 10. Following release of a load, fluid 60 will travel back through valve 58, and membrane 52 will return to its pre-load configuration. Upon subsequent applications of a load, the cycle will repeat. Similar to load absorption units described above, a plurality of units 50 will collectively perform in a similar fashion, thereby performing the function of a healthy native disc nucleus.

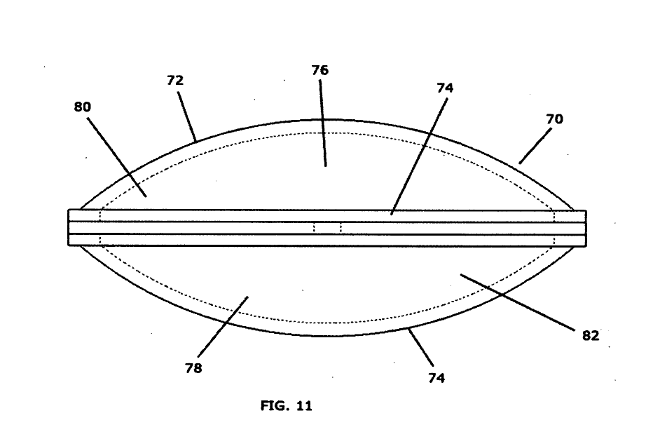

Another alternative embodiment according to the invention is illustrated in FIG. 11. Load absorption unit 70 comprises relatively compliant membrane 72, relatively rigid membrane 74, relatively compliant plate 73 disposed within its substantially hollow interior and defining first chamber 76 and second chamber 78. First chamber 76 comprises fluid 80, and second chamber 78 comprises gas 82.

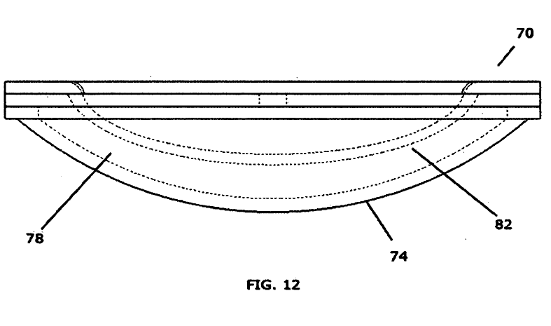

As illustrated in FIG. 12, following application of a load, membrane 72 is compressed, forcing fluid 80 against plate 73, which is forced into second chamber 78, thereby compressing gas 82. Following release of a load, unit 70 returns to its unstressed configuration (as illustrated in FIG. 11), and the cycle can repeat. A plurality of units 70, when enveloped by a membrane (such as, for example, membrane 12 of FIG. 1 or membrane 32 of FIG. 5) will collectively perform as described throughout cycles of application and removal of a load, and will thereby perform the functions of a healthy native disc nucleus. Alternatively, a unit similar to the foregoing, but larger, may function alone as an artificial disc nucleus. Also in the alternative, a unit may further comprise one or more sensors and activation mechanisms that respond to a sensor. Such a unit may be filled with a responsive hydrogel or EPAM which may undergo changes in response to an activation mechanism similar to that described below.

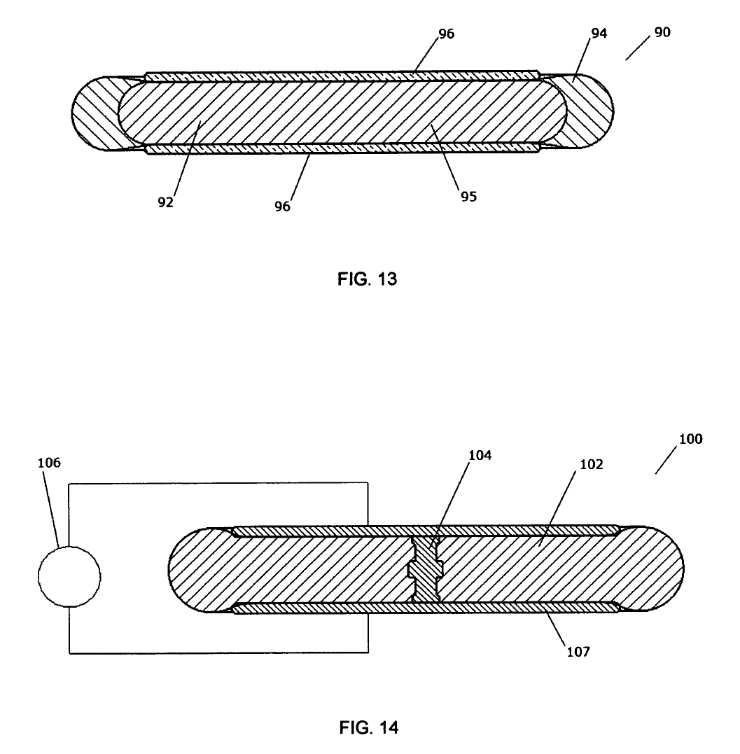

Turning now to FIG. 13, yet another embodiment according to the invention is described. Artificial nucleus 90, shown in a cross sectional side view, comprises first reservoir 92 and second reservoir 94. First reservoir 92 may be filled with a responsive hydrogel 95, or alternatively, EPAM or other suitable material. Second reservoir 94 may be filled with a responsive hydrogel, EPAM, water, or other suitable material. Nucleus 90 further comprises one or more activation plates 96. Activation plates 96 may be constructed of suitable materials and of suitable configuration to receive an electrical, thermal, radiofrequency, pH, chemical, or other stimulus. Such stimulus will then induce a selected response in hydrogel 95 (or EPAM).

Artificial nucleus 90 may be coupled with a control system (not pictured) for delivery of the particular stimulus which induces the selected response in hydrogel 95 (or EPAM). Upon activation by a stimulus, hydrogel 95 may draw water from second reservoir 94 and may swell or otherwise undergo a desired change in configuration. In one example, hydrogel 95 and nucleus 90 undergo some compacting or shrinking as a result of the application of a load, such as, for example, throughout the day of a subject. Upon activation by a stimulus, for example, at the end of the day of a subject, hydrogel 95 may then swell and return to its pre-load configuration. Nucleus 90 will thereby mimic the behavior of a healthy native disc nucleus, which may decrease in height and/or volume during the day, and hydrate, and return to normal height during rest.

Alternatively, first reservoir 92 may comprise EPAM, and an electrical potential may be created upon application of a load, which may be utilized to activate EPAM and/or a responsive hydrogel to swell or otherwise change configuration And as yet another alternative, second reservoir 94 may comprise EPAM, which upon compression creates an electrical charge, which then flows to control plates 96 and activates responsive hydrogel 95 to undergo a desired change in configuration.

FIG. 14 illustrates yet another alternative embodiment according to the invention which is similar to that described in relation to FIG. 13, with some modifications. Artificial nucleus 100 comprises EPAM 102 within its interior. Artificial nucleus 100 further comprises strain gauge 104, which may provide feedback to control system 106. As one example, strain gauge 104 may provide feedback to control system 106 that will trigger one or more activation plates 106 to deliver a desired stimulus (such as, for example, electrical, radiofrequency, pH, chemical, or other stimulus) to EPAM 102. As another example, if nucleus 100 comprises EPAM, an electrical potential may be created upon application of a load, and may be stored within a battery or capacitor, or may activate EPAM or a responsive hydrogel. It will be appreciated by one skilled in the art that variations may be made in the configuration of the reservoirs and filling material without departing from the scope of the invention.

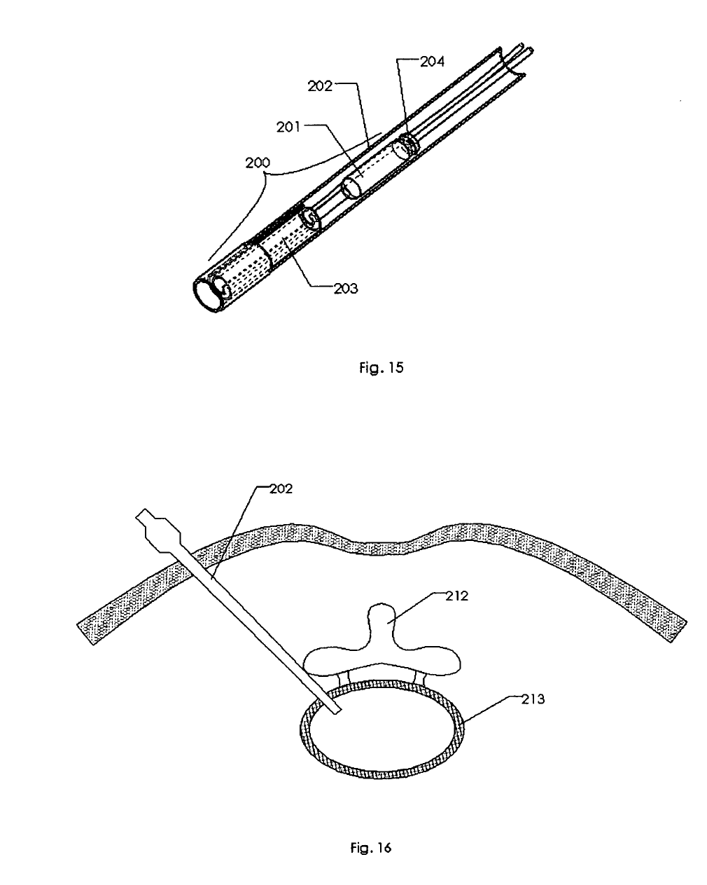

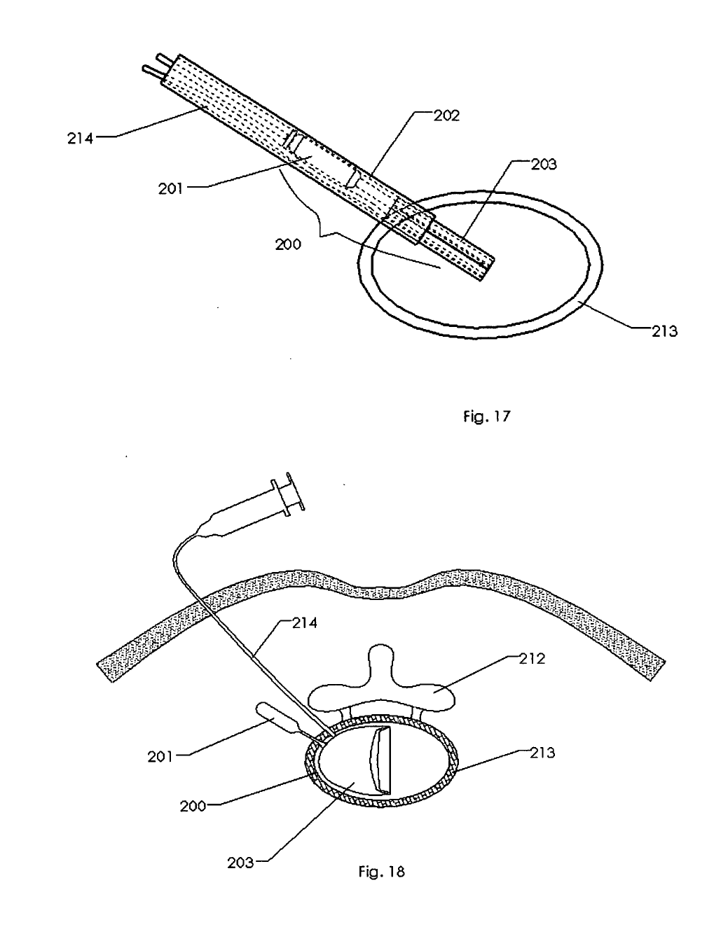

Delivery and deployment of an alternative embodiment according to the invention following a posterior-lateral approach is illustrated in FIGS. 15-20. In FIG. 15, artificial nucleus 200, having body 203 and shock absorber 201, is shown in “see-through” mode in its delivery configuration within trocar or cannula 202. Pusher 204 will force nucleus 200 through cannula 202. The position of cannula 202 in relation to vertebra 212 and native disc 213 of a subject prior to delivery and deployment is illustrated in FIG. 16. The native disc nucleus and, if desired, disc annulus may be removed according to a suitable procedure prior to delivery of artificial nucleus 200. FIG. 17 illustrates a more detailed view of the delivery position of cannula 202 at a later step in the sequence of delivery of nucleus 200. Body 203 of nucleus 200 is shown emerging from cannula 202, illustrated in “see-through” mode. Cannula 202 may be of any number of desired of designs, including having separate lumens for the housing and delivery of removable fill tube 214 and other elongate members useful in the percutaneous delivery of nucleus 200.

FIG. 18 illustrates the delivery and deployment of artificial nucleus 200 following a step in which dampening member or shock absorber 201 has been positioned (through an opening through the native disc annulus if the native annulus has been left intact), fill material is entering nucleus 200 via removable fill tube 214. Nucleus body 203 is in the process of unrolling to fill the disc space of the subject.

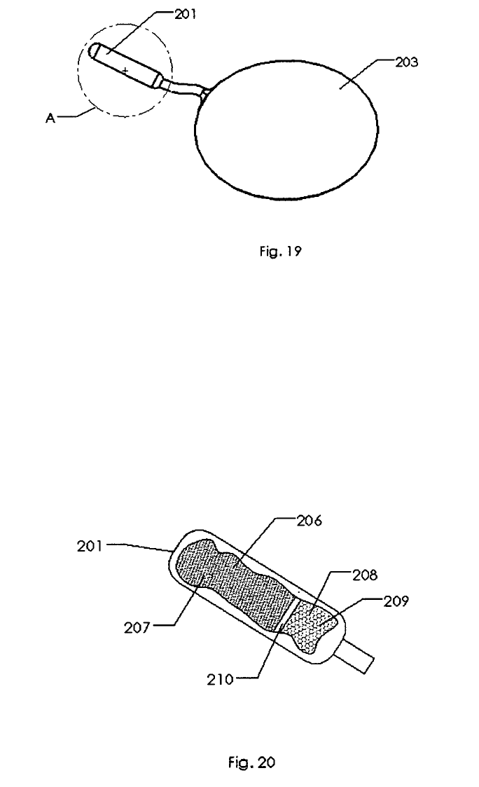

FIG. 19 illustrates nucleus 200 by itself in a deployed configuration. FIG. 20 illustrates in cross section detail of area A of FIG. 19, which depicts shock absorber 201. Shock absorber 201 comprises first chamber 206 and second chamber 208 divided by partition 210. First chamber 206 comprises carbon dioxide or other suitable gas 207 and second chamber 208 comprises hydrogel 209. Hydrogel 209 may or may not be responsive to stimuli similar to responsive hydrogel 95 described above in relation to FIG. 13. Alternatively, first chamber 206 or second chamber 208 may comprise EPAM. The interior of body 203 of nucleus 200 also comprises a hydrogel, which may or may not be a responsive hydrogel and may alternatively comprise EPAM, and is in fluid communication with shock absorber 201. Nucleus 200 may comprise a valve disposed in its interior between body 203 and shock absorber 201.

Upon application of a load to artificial nucleus 200, hydrogel 209 flows from body 203 (through a valve, if desired) to shock absorber 201. Partition 209 is forced against gas 207, thereby compressing gas 207. Following release of a load, hydrogel 209 can return to body 203, and nucleus 200 can return to its equilibrium force. Thereafter the cycle may repeat.

In an alternative embodiment, shock absorber 201 may house a control system (not pictured) having, for example, a pressure sensor or strain gauge, electronics and a power source. In response to the application of a load, a control system may supply current to activate a responsive hydrogel to undergo a desired change in configuration, such as, for example, swelling. And as yet another alternative, one or more chambers may comprise EPAM, in which an electrical potential is created upon application of a load, which may then be utilized to activate a responsive hydrogel. It will be appreciated by one skilled in the art that the configuration of chambers and fill material may be rearranged in innumerable ways without departing from the scope of the invention.

While all of the foregoing embodiments can most advantageously be delivered in a minimally invasive, percutaneous manner, the foregoing embodiments may also be implanted surgically. Further, while particular forms of the invention have been illustrated and described above, the foregoing descriptions are intended as examples, and to one skilled in the art it will be apparent that various modifications can be made without departing from the spirit and scope of the invention.

We claim:

1. An artificial disc or disc nucleus comprising a first membrane and a second membrane wherein said first membrane defines a first chamber comprising a first fluid and said second membrane defines one or more dampening members comprising a second fluid.

2. The artificial disc or disc nucleus according to claim 1 wherein said first and second membranes are substantially impermeable.

3. The artificial disc or disc nucleus according to claim 2 wherein said second membrane comprises a metallized coating.

4. The artificial disc or disc nucleus according to claim 1 further comprising a third membrane wherein said third membrane is permeable and defines a third chamber substantially surrounding said one or more dampening members.

5. The artificial disc or disc nucleus according to claim 1 wherein said second fluid comprises one or more compressible gases.

6. The artificial disc or disc nucleus according to claim 1 said first fluid or said second fluid comprises a responsive hydrogel.

7. The artificial disc or disc nucleus according to claim 1 wherein said first fluid or said second fluid comprises EPAM.

8. An artificial disc or disc nucleus comprising one or more activation members and one or more chambers comprising one or more fluids in communication with one or more activation members.

9. The artificial disc or disc nucleus according to claim 8 wherein one or more of said fluids comprises a responsive hydrogel.

10. The artificial disc or disc nucleus according to claim 8 wherein one or more of said fluids comprises EPAM.

11. The artificial disc or disc nucleus according to claim 8 further comprising one or more sensors for detecting a change in one or more physical or chemical characteristics of one or more of said fluids.

12. The artificial disc or disc nucleus according to claim 11 wherein said one or more physical or chemical characteristics is selected from the list consisting of volume, compression, density, strain, temperature, pH, salts concentration, electrical potential, and hydration.

13. The artificial disc or disc nucleus according to claim 8 further comprising a control system in communication with said artificial disc or disc nucleus wherein said control system delivers one or more stimuli to said artificial disc or disc nucleus.

14. The artificial disc or disc nucleus according to claim 13 wherein said one or more stimuli is selected from the list consisting of electrical charge, radiofrequency, ultrasound, and heat.

15. The artificial disc or disc nucleus according to claim 1 wherein said one or more dampening members comprises one or more valves for regulating the flow of one or more fluids within said dampening member.

16. The artificial disc or disc nucleus according to claim 1 wherein said first membrane defines a body, said disc or disc nucleus further comprising one or more valves disposed between said body and said one or more dampening members for regulating the flow of one or more fluids between said body and said one or more dampening members.

17. The artificial disc or disc nucleus according to claim 16 wherein said one or more dampening members comprises one or more chambers.

18. The artificial disc or disc nucleus according to claim 1 wherein said second membrane comprises one or more compliant regions and one or more rigid regions.

19. A method of manufacture of an artificial disc or disc nucleus comprising the steps of:
preparing a first polymeric membrane;
forming a body and one or more dampening members from said membrane, where said body comprises an interior.

20. The method according to claim 19 with the added step of introducing said one or more dampening members into the interior of said body.

21. The method according to claim 19 wherein one or more said dampening members is prepared according to a method comprising the steps of:
forming an enclosed member from said first membrane;
introducing a compressible gas in a supercritical state into said member;
allowing said compressible gas to return to ambient temperature to form a dampening member.

22. The method according to claim 19 with the added steps of preparing a second polymeric membrane;
substantially enclosing said one or more dampening members with said second membrane, where said second membrane is permeable.

23. The method according to claim 19 with the additional step of introducing one or more fluids into said body.

24. The method according to claim 19 with the additional step of providing a valve within said dampening member.

25. The method according to claim 19 wherein said second membrane comprises one or more compliant regions and one or more rigid regions.

26. The method according to claim 19 wherein one or more of said fluids comprises a responsive hydrogel.

27. The method according to claim 19 wherein one or more of said fluids comprises EPAM.

28. The method according to claim 21 wherein said membrane is metallized either prior to or subsequent to the introduction of said compressible gas.

29. The method according to claim 19 with the additional steps of forming said dampening member exterior to said body;
providing a partition between said body and said dampening member; and
introducing a first and second fluid into said body and said dampening member.

30. The method according to claim 29 with the additional steps of providing a control system in communication with said artificial disc or disc nucleus.

31. A method of manufacture of an artificial disc or disc nucleus comprising the steps of:
providing a body comprising one or more chambers and one or more activation members in communication with one or more of said chambers;
introducing one or more fluids into said one or more chambers, wherein one or more of said fluids comprises a responsive hydrogel or EPAM.

32. The method according to claim 26 with the additional step of providing one or more sensors and a control system in communication with said artificial disc or disc nucleus.
